(19) United States
(12) Patent Application Publication
(10) Pub. No.: US 2005/0222009 A1
(43) Pub. Date: Oct. 6, 2005

(54) Dual phase - PNA conjugates for the delivery of PNA through the blood brain barrier

(76) Inventors: Itschak Lamensdorf, Modlin (IL); Jehoshua Katzhendler, Jerusalem (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/962,659

(22) Filed: Oct. 13, 2004

Related U.S. Application Data
(60) Provisional application No. 60/510,137, filed on Oct. 14, 2003.

(51) Int. Cl. A61K 48/00; C07K 14/00; C12N 15/85

(52) U.S. Cl. 514/7; 435/455; 530/352

(57) ABSTRACT

The invention provides molecule comprising a nucleic acid, a peptide ligand, which binds to a specific receptor and a positively charge peptide moiety with lysosomatic properties, useful in the delivery of a nucleic acid across a cellular membrane. The invention further relates to the use of these compounds for the delivery of a nucleic acid to the brain across the blood brain barrier for diagnostic and therapeutic applications

### CROSS REFERENCE DATA

This Application claims the priority of U.S. Provisional Application No. 60/510,137 filed Oct. 14, 2003.

### FIELD OF THE INVENTION

The invention provides compounds comprising a nucleic acid, a peptide ligand which binds to a specific receptor and a positively charge peptide moiety with lysosomatic properties, useful in the delivery of a nucleic acid across a cellular membrane. The invention further relates to the use of these compounds for the delivery of a nucleic acid into the brain for diagnostic and therapeutic applications.

### BACKGROUND OF THE INVENTION

The protective function of the blood-brain barrier becomes an important problem in the treatment of neurological diseases. The exclusion of blood-borne foreign substances also results in the exclusion of a large number of potentially therapeutic agents from the brain. The blood brain barrier (BBB) is a very complex endothelial interface separating the brain from the blood compartment and impeding the delivery of 98% of drugs to the brain. The BBB consists of a monolayer of polarized endothelial cells connected by complex tight junctions.

For small drug molecules (>500 Da) the primary factors influencing BBB permeability are molecular weight, hydrogen bonding, lipophilicity and the ability to bind to plasma proteins. In addition, small molecules can cross the BBB utilizing nutrient transporters (for glucose, amino acids, organic acids, or adenosine). However, nutrient transporters in the BBB can deliver only low molecular weight substrates. Several strategies such as drugs lipidization (addition of lipid-like molecules to the drug) and linkage to chemical delivery systems (CDS), are known in the art for the delivery of compounds through the BBB. Unfortunately, these strategies can be used only for the delivery of relatively small molecule.

Large molecules can across the capillary barrier via receptor mediated transport mechanisms (RMT) such as the insulin receptor or the transferrin receptor, or via absorptive mediated transcytosis (AMT), such as cationized albumin.

It is known in the art that peptides can cross the blood brain barrier via receptor mediated mediated transcytosis. This process involved binding of the receptor and peptide at one side of the BBB (e.g. the luminal membrane), translocation of the receptor-peptide complex through the cytoplasm, and dissociation of the peptide from the receptor on the externalsurface of the abluminal membrane.

Antisense drugs are small complementary strands of DNA (oligonucleotides; ODNs) designed to bind to a specific sequence of nucleotides in the mRNA target, thus inhibiting production of the encoded protein. However, due to their low biomembrane permeability and their relatively rapid degradation oligonucleotides are generally considered to be of limited therapeutic value. To improve their therapeutic applications, the backbone of these antisense compounds has been chemically modified. The third generation of antisense chemistry is the polyamide (peptide) nucleic acid (PNA) surrogates. PNAs are the first successful substitutes of ODNs that have displayed equal or better binding affinity than natural DNA or RNA antisense-based drugs. PNAs are apolar molecule with poor bioavaliability properties. Therefore, unmodified/naked PNA molecules pass poorly through the cell membrane and do not have useful therapeutic applications. Both ODNs and PNAs cannot cross the endothelial cellular membrane of the BBB effectively, thus, preventing the possible use of these technologies in developing drugs for CNS disorders.

### SUMMARY OF THE INVENTION

In one embodiment, the invention provides a molecule represented by any one of the formulas I-IV:
[(L)ₜ-(N)ᵣ-(H)_{q}-(P)ₛ]ₓ I.
[(L)ₜ-(H)_{q}-(N)ᵣ-(P)ₛ]ₓ II.
[(P)ₛ-(N)ᵣ-(H)_{q}-(L)ₜ]ₓ III.
[(P)ₛ-(H)_{q}-(N)ᵣ-(L)ₜ]ₓ IV.
wherein N is a nucleic acid sequence in a length of 1-100 bases, L is a peptide ligand which binds to a specific receptor, P is a positively charge moiety and H is a hydrophobic moiety; and
wherein r is an integer of 1-25, t is an integer of 1-50, s is an integer of 1-25, q is an integer of 0-20, and x is an integer of 1-20.

In one embodiment, the invention provides a molecule represented by any one of the formulas V-VIII:
[(L)ₜ-(PNA)ᵣ-(H)_{q}-(P)ₛ]ₓ V.
[(L)ₜ-(H)_{q}-(PNA)ᵣ-(P)ₛ]ₓ VI.
[(P)ₛ-(PNA)ᵣ-(H)_{q}-(L)ₜ]ₓ VII.
[(P)ₛ-(H)_{q}-(PNA)ᵣ-(L)ₜ]ₓ VIII.
wherein the length of said PNA sequence is 1-100 bases, L is a peptide ligand which binds to a specific receptor, P is a positively charge moiety, and H is a hydrophobic moiety; and
wherein r is an integer of 1-25, t is an integer of 1-50, s is an integer of 0-25, q is an integer of 0-20 and x is an integer of 1-20.

In one embodiment, the invention further provides a method for delivering a molecule across a cellular membrane.

In one embodiment, the invention further provides a method for delivering a nucleic acid sequence to the brain across the blood brain barrier.

### BRIEF DESCRIPTION OF THE DRAWINGS

The invention will be understood and appreciated more fully from the following detailed description taken in conjunction with the appended drawings in which:

FIG. 1 demonstrates the uptake of conjugated PNAs into PC12 cells.

FIG. 2 demonstrates the Neural Red uptake into PC12 cells following 48 h incubation with conjugated PNAs.

FIG. 3 demonstrates fluorescence images of bEND3 cells incubated with fluorescence-labelled peptide-PNA conjugated or fluorescence-labelled unmodified PNA.

FIG. 4 demonstrates the uptake of conjugated PNAs into human neuronal NMB cells. demonstrates the uptake of conjugated PNAs into PC12 cells.

FIGS. 5(a) and (b) demonstrate the in-vivo brain uptake of conjugated PNAs.

### DETAILED DESCRIPTION OF THE INVENTION

In one embodiment, the invention provides a molecule represented by any one of the formulas I-IV:
[(L)ₜ-(N)ᵣ-(H)_{q}-(P)ₛ]ₓ V.
[(L)ₜ-(H)_{q}-(N)ᵣ-(P)ₛ]ₓ VI.
[(P)ₛ-(N)ᵣ-(H)_{q}-(L)ₜ]ₓ VII.
[(P)ₛ-(H)_{q}-(N)ᵣ-(L)ₜ]ₓ VIII.
wherein N is a nucleic acid sequence in a length of 1-100 bases, L is a peptide ligand which binds to a specific receptor and P is a positively charge moiety and H is a hydrophobic moiety; and
wherein r is an integer of 1-25, t is an integer of 1-50, s is an integer of 1-25, q is an integer of 0-20, and x is an integer of 1-20.

In one embodiment, the invention provides a molecule represented by any one of the formulas V-VIII:
[(L)ₜ-(PNA)ᵣ-(H)_{q}-(P)ₛ]ₓ V.
[(L)ₜ-(H)_{q}-(PNA)ᵣ-(P)ₛ]ₓ VI.
[(P)ₛ-(PNA)ᵣ-(H)_{q}-(L)ₜ]ₓ VII.
[(P)ₛ-(H)_{q}-(PNA)ᵣ-(L)ₜ]ₓ VIII.
wherein the length of said PNA sequence is 1-100 bases, L is a peptide ligand which binds to a specific receptor and P is a positively charge moiety and H is a hydrophobic moiety; and
wherein r is an integer of 1-25, t is an integer of 1-50, s is an integer of 0-25, q is an integer of 0-20 and x is an integer of 1-20.

In one embodiment of the invention, N is a nucleic acid sequence in a length of 1-100 bases. In another embodiment of the invention, N is a nucleic acid sequence in a length of 1-10 bases. In another embodiment of the invention, N is a nucleic acid sequence in a length of 1-20 bases. In another embodiment of the invention, N is a nucleic acid sequence in a length of 10-20 bases. In another embodiment of the invention, N is a nucleic acid sequence in a length of 20-30 bases. In another embodiment of the invention, N is a nucleic acid sequence in a length of 30-40 bases. In another embodiment of the invention, N is a nucleic acid sequence in a length of 40-50 bases. In another embodiment of the invention, N is a nucleic acid sequence in a length of 50-100 bases.

In one embodiment of the invention, the length of the PNA sequence is 1-100 bases. In another embodiment of the invention, the length of the PNA sequence is 1-10 bases. In another embodiment of the invention, the length of the PNA sequence is 1-20 bases. In another embodiment of the invention, the length of the PNA sequence is 10-20 bases. In another embodiment of the invention, the length of the PNA sequence is 20-30 bases. In another embodiment of the invention, the length of the PNA sequence is 30-40 bases. In another embodiment of the invention, the length of the PNA sequence is 40-50 bases In another embodiment of the invention, the length of the PNA sequence is 50-100 bases.

In one embodiment of the invention, q is an integer of 0-20. In another embodiment of the invention, q is an integer of 2-10. In another embodiment of the invention, q is an integer of 6-16. In another embodiment of the invention, q is 8. In another embodiment of the invention, q is 9. In another embodiment of the invention, q is 0.

In one embodiment of the invention, r is an integer of 0-20. In another embodiment of the invention, r is an integer of 1-10. In another embodiment of the invention, r is an integer of 10-20. In another embodiment of the invention, r is an integer of 2-5.

In one embodiment of the invention, s is an integer of 0-25. In another embodiment of the invention, s is an integer of 2-15. In another embodiment of the invention, s is an integer of 2-6. In another embodiment of the invention, s is 4. In another embodiment of the invention, s is 0.

In one embodiment of the invention, t is an integer of 1-50. In another embodiment of the invention, t is an integer of 5-25. In another embodiment of the invention, t is an integer of 10-15.

In one embodiment of the invention, x is an integer of 1-20. In another embodiment of the invention, x is an integer of 2-15. In another embodiment of the invention, s is an integer of 5-10.

In one embodiment of the invention, the nucleic acid sequence is a mRNA. In another embodiment the nucleic acid sequence is a cDNA. In another embodiment the nucleic acid sequence is a DNA. In another embodiment the nucleic acid sequence is a DNA analog. In another embodiment the nucleic acid sequence is a PNA In another embodiment the nucleic acid sequence is a PNA morpholino. In another embodiment the nucleic acid sequence is an aminoethylprolyl (aep) PNA. In another embodiment the nucleic acid sequence is a pyrrolidinyl PNA. In another embodiment the nucleic acid sequence is an oligonucleotide. In another embodiment the nucleic acid sequence is an oligonucleotide analog. In another embodiment the nucleic acid sequence is a ribozyme. In another embodiment the nucleic acid sequence is an RNAi. In another embodiment of the invention, the nucleic acid sequence is a PNA.

In one embodiment of the invention, the nucleic acid sequence is an antisense. In another embodiment of the invention, the nucleic acid sequence is an antigene. In another embodiment of the invention, the nucleic acid sequence is a decoy function. In one embodiment of the invention, the nucleic acid sequence is neutral. In another embodiment the nucleic acid sequence is negatively charged. In one embodiment of the invention, nucleic acid sequence is in antisense orientation to an endogenous sequence.

In one embodiment of the invention, the PNA sequence is an antisense. In another embodiment of the invention, the PNA sequence is an antigene. In another embodiment of the invention, the PNA sequence is a decoy function. In one embodiment of the invention, the PNA sequence is neutral. In another embodiment the PNA sequence is negatively charged. In one embodiment of the invention, PNA sequence is in antisense orientation to an endogenous sequence.

The term “nucleotide” describes a subunit of DNA or RNA consisting of a nitrogenous base (adenine, guanine, thymine, or cytosine in DNA; adenine, guanine, uracil, or cytosine in RNA), a phosphate molecule, and a sugar molecule (deoxyribose in DNA and ribose in RNA). Thousands of nucleotides are linked to form a DNA or RNA molecule. The term nucleotide further describes protected guanine; pseudo-guanine or protected pseudo-guanine (2,6-diaminopurine); protected adenine; protected cytosine; pseudo-cytosine or protected pseudo-cytosine, pseudo-isocytosine or protected pseudo-isocytosine; protected uracil.

The term “oligonucleotide” describes a molecule usually composed of 25 or fewer nucleotides.

The term “antisense” describes a nucleic acid sequence that has a sequence exactly opposite to an mRNA molecule made by the body; binds to the mRNA molecule to prevent a protein from being made.

The term “peptide nucleic acids” (PNAs) refers to molecules that in certain respects are similar to oligonucleotide analogs however in other very important respects their structure is very different. In peptide nucleic acids, the deoxyribose phosphate backbone of oligonucleotides has been replaced with a backbone more akin to a peptide than a sugar phosphodiester. Each subunit has a naturally occurring or non-naturally occurring base attached to this backbone. A non-limiting example is a backbone constructed of repeating units of N-(2-aminoethyl)glycine or analogues thereof having a nucleobase attached thereto via a linker such as a carboxymethyl moiety or analogues thereof to the nitrogen atom of the glycine portion of the unit The units are coupled together via amide bonds formed between the carboxyl group of the glycine moiety and the amine group of the aminoethyl moiety. The nucleobase can be one of the four common nucleobases of nucleic acids or they can include other natural or synthetic nucleobases. Due to the radical deviation from the deoxyribose backbone, these molecules were named peptide nucleic acids.

The term “antigene” refers to molecules, which bind to double-stranded DNA. Antigenes can enhance or inhibit gene expression in cells.

In one embodiment, the invention provides an improved method for the delivery of PNA-based nucleic acid whereas the PNA is used a spacer between the peptide ligand and the positively charged moiety. Thus the chimera can cross the blood brain barrier either via RMT or AMT. Thus, in one embodiment of the invention the PNA/nucleic acid is functioning as a spacer diminishing steric/electrostatic interaction between the two peptide moiety. In another embodiment the PNA/nucleic acid is used as an antisense moiety, an antigene moiety or a gene modulator moiety. In another embodiment of the invention, the PNA/nucleic acid molecule is used as an apolar peptide-like moiety in an amphiphlic brain vector.

In one embodiment, the invention provides an improved method for delivery of PNA-based nucleic acid through the BBB following addition of hydrophobic moiety to the PNA-peptide chimera. The resulted compound can cross the BBB as a result of its ampiphilic structure, as a result of receptor mediated transcytosis or both mechanisms.

In one embodiment of the invention, the peptide ligand binds a receptor to transferrin. In another embodiment of the invention, the peptide ligand binds a receptor to insulin. In another embodiment of the invention, the peptide ligand binds a receptor to insulin growth factor. In another embodiment of the invention, the insulin growth factor is an insulin growth factor-I. In another embodiment of the invention, the insulin growth factor is an insulin growth factor-II. In another embodiment of the invention, the peptide ligand binds a receptor to leptin. In another embodiment of the invention, the peptide ligand binds a receptor is HAIYPRH (SEQ ID No. 1). In another embodiment of the invention, the peptide ligand binds a receptor is THRPPMWSPVWP (SEQ ID No. 2).

In one embodiment of the invention, the hydrophobic moiety is a nucleic acid. In another embodiment of the invention, the hydrophobic moiety is a nucleic acid analog. In another embodiment of the invention, the hydrophobic moiety is a hydrophobic peptide. In another embodiment of the invention, the hydrophobic moiety is a lipid acid. In another embodiment of the invention, the hydrophobic moiety is a lipid molecules. In another embodiment of the invention, the hydrophobic moiety is octanol. In another embodiment of the invention, the hydrophobic moiety is cholesterol. In another embodiment of the invention, the hydrophobic moiety is a hydrophobic peptide protecting group. In another embodiment of the invention, the hydrophobic moiety is adamantine. In another embodiment of the invention, the hydrophobic moiety is pyrene. In another embodiment of the invention, the hydrophobic moiety is eicosenoic acid. In another embodiment of the invention, the hydrophobic moiety is a C(₆-₁₆) glyceride lipid. In another embodiment the hydrophobic moiety is phenoxazine. In another embodiment of the invention, the hydrophobic moiety is a DMT group. In another embodiment of the invention, the hydrophobic moiety is cholenic acid. In another embodiment of the invention, the hydrophobic moiety is lithocholic acid. In another embodiment of the invention, the hydrophobic moiety is myristic acid. In another embodiment of the invention, the hydrophobic moiety is palmitic acid. In another embodiment of the invention, the hydrophobic moiety is a heptadecyl group. In another embodiment the hydrophobic moiety is hexadecylglycerol. In another embodiment of the invention, the hydrophobic moiety is a geranyloxyhexyl group. In another embodiment the hydrophobic moiety is hexadecylamine. In another embodiment the hydrophobic moiety is dihydrotestosterone. In another embodiment of the invention, the hydrophobic moiety is 1-pyrene butyric acid. In another embodiment of the invention, the hydrophobic moiety is alkanoic acid. In another embodiment of the invention, the hydrophobic moiety is alkanol. In another embodiment the hydrophobic moiety is and any derivatives of the above mentioned moieties. In one embodiment of the invention, the alkanoic acid is represented by the structure R—(CH₂)n-COOH, wherein n is an integer of 1-20 and R is a linear or branched alkyl. In another embodiment n is an integer of 6-16. In one embodiment of the invention, the alkanol is represented by the structure R—(CH₂)n-OH, wherein n an integer of 1-20 and R is a linear or branched alkyl. In another embodiment of the invention, n is an integer of 6-16. In one embodiment of the invention, the lipid acid is undecanoic acid. In another embodiment of the invention, the lipid acid is docosahexanenonic acid. In one embodiment of the invention, the hydrophobic peptide protecting group is Fmoc. In another embodiment of the invention, the hydrophobic peptide protecting group is Tboc.

As contemplated herein, an “alkyl” group refers to a saturated aliphatic hydrocarbon, including straight-chain, branched-chain and cyclic alkyl groups. In one embodiment of the invention, the alkyl group has 1-4 carbons. In another embodiment of the invention, the alkyl group is a methyl group. In another embodiment of the invention, the alkyl group is an ethyl group. In another embodiment of the invention, the alkyl group is a propyl group. In another embodiment of the invention, the alkyl group is a butyl group. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

In one embodiment of the invention, the molecule is designed to have a positively charged moieties conjugated to the N or C terminal of the modified-PNA in accordance with the invention.

In one embodiment of the invention, the positively charge moiety is a nucleic acid sequence. In another embodiment of the invention, the positively charge moiety a nucleic acid analog. In another embodiment of the invention, the positively charge moiety a PNA. In another embodiment of the invention, the positively charge moiety is a positively charge peptide. In another embodiment of the invention, the positively charge moiety is a peptidomimetic. In another embodiment of the invention, the positively charge moiety is a polycations. In another embodiment of the invention, the positively charge moiety is a histidine. In another embodiment of the invention, the positively charge moiety is an imidazole group. In another embodiment of the invention, the positively charge moiety is 2-O-aminopropyl. In another embodiment of the invention, the positively charge moiety is 2-O-dimethylaminopropyl. In another embodiment of the invention, the positively charge moiety is 2-O-imidazolyl-ethyl. In another embodiment of the invention, the positively charge moiety is 2-O-aminoethylamino-oxyethyl. In another embodiment of the invention, the positively charge moiety is 2-dimethylaminoethyl-oxyethyl. In another embodiment of the invention, the positively charge moiety is and any derivative of the above mentioned moieties. In another embodiment of the invention, the positively charge moiety is arginine. In another embodiment of the invention, the positively charge moiety is D-arginine. In another embodiment of the invention, the positively charge moiety is polyarginine. In another embodiment of the invention, the positively charge moiety is polyamine. In another embodiment of the invention, the positively charge moiety is guanidine. In another embodiment of the invention, the polyamine is spermine. In another embodiment of the invention, the polyamine is spermidine. In another embodiment of the invention, the polyamine is putricine.

In one embodiment of the invention, the positively charge moiety is a cationic peptide. In another embodiment of the invention, the cationic peptide is CHK₆HC (SEQ ID No. 3).

In one embodiment of the invention, the positively charge moiety is CK4HK₃C (SEQ ID No. 4). In another embodiment of the invention, the positively charge moiety is CHK₆HC (SEQ ID No. 3). In another embodiment of the invention, the positively charge moiety is CHK₃HK₂HC (SEQ ID No. 5). In another embodiment of the invention, the positively charge moiety is C(HK)₄C (SEQ ID No. 6). In another embodiment of the invention, the positively charge moiety is CHKHKHHKHC (SEQ ID No. 7).

In one embodiment of the invention, the PNA sequence is CCGCTCCG (SEQ ID No. 8). In another embodiment of the invention, the PNA sequence is CAT GGT GGA CGT (SEQ ID No. 9). In another embodiment of the invention, the PNA sequence is CTT TCT CCT TTT CC (SEQ ID No. 10). In another embodiment of the invention, the PNA sequence is TACTCATGGGCACACT (SEQ ID No. 11). In another embodiment of the invention, the PNA sequence is TTT GCT CTT ACT CAT (SEQ ID No. 12). In another embodiment of the invention, the PNA sequence is GCAT (SEQ ID No. 13). In another embodiment of the invention, the PNA is an (aminoethylprolyl (aep) PNA)₁₋₂₀

In one embodiment of the invention, the peptide ligand, the hydrophobic moiety, the PNA/nucleic acid sequence and the positively charge moiety are linked to each other directly via peptide bonds.

In one embodiment, the invention provides a molecule comprising CHK6HC-(PNA)ᵣ-HAIYPRH (SEQ ID No. 14). In one embodiment, the invention provides a molecule comprising CHK₆HC-(PNA)ᵣ-THRPPMWSPVWP (SEQ ID No. 15). In one embodiment, the invention provides a molecule comprising CK₄HK₃C-(PNA)ᵣ-HAIYPRH (SEQ ID No. 16). In one embodiment, the invention provides a molecule comprising CK₄HK₃C-(PNA)ᵣ-THRPPMWSPVWP (SEQ ID No. 17). In one embodiment, the invention provides a molecule comprising CHK₃HK₂HC-(PNA)ᵣ-HAIYPRH (SEQ ID No. 18). In one embodiment, the invention provides a molecule comprising CHK₃HK₂HC-(PNA)ᵣ-THRPPMWSPVWP (SEQ ID No. 19). In one embodiment, the invention provides a molecule comprising C(HK)₄C-(PNA)ᵣ-HAIYPRH (SEQ ID No. 20). In one embodiment, the invention provides a molecule comprising C(HK)₄C-(PNA)ᵣ-THRPPMWSPVWP (SEQ ID No. 21). In one embodiment, the invention provides a molecule comprising CHKHKHHKHC-(PNA)ᵣ-HAIYPRH (SEQ ID No. 22). In one embodiment, the invention provides a molecule comprising CHKHKHHKHC-(PNA)ᵣ-THRPPMWSPVWP (SEQ ID No. 23). In one embodiment of the invention r is 5-25. In another embodiment r is 5-10. In another embodiment r is 10-20.

In one embodiment, the invention provides a molecule comprising-HAIYPRH-(PNA)ᵣ-CHK₆HC (SEQ ID No. 24). In one embodiment, the invention provides a molecule comprising THRPPMWSPVWP-(PNA)ᵣ-CHK₆HC (SEQ ID No. 25). In one embodiment, the invention provides a molecule comprising HAIYPRH-(PNA)ᵣ-CK₄HK₃C (SEQ ID No. 26). In one embodiment, the invention provides a molecule comprising THRPPMWSPVWP-(PNA)ᵣ-CK₄HK₃C (SEQ ID No. 27). In one embodiment, the invention provides a molecule comprising HAIYPRH-(PNA)ᵣ-CHK₃HK₂HC (SEQ ID No. 28). In one embodiment, the invention provides a molecule comprising THRPPMWSPVWP-(PNA)ᵣ-CHK₃HK₂HC (SEQ ID No. 29). In one embodiment, the invention provides a molecule comprising HAIYPRH-(PNA)ᵣ-C(HK)₄C (SEQ ID No. 30). In one embodiment, the invention provides a molecule comprising THRPPMWSPVWP-(PNA)ᵣ-C(HK)₄C (SEQ ID No. 31). In one embodiment, the invention provides a molecule comprising HAIYPRH-(PNA)ᵣ-CHKHKHHKHC (SEQ ID No. 32). In one embodiment, the invention provides a molecule comprising THRPPMWSPVWP-(PNA)ᵣ-CHKHKHHKHC (SEQ ID No. 33). In one embodiment of the invention r is 5-25. In another embodiment r is 5-10. In another embodiment r is 10-20.

In one embodiment, the invention provides a molecule comprising CHK₆HC-TTT GCT CTT ACT CAT-THRPPMWSPVWP (SEQ ID No. 34). In one embodiment, the invention provides a molecule comprising CHK₆HC-TTT GCT CTT ACT CAT-HAIYPRH (SEQ ID No. 35). In one embodiment, the invention provides a molecule comprising THRPPMWSPVWP-TTT GCT CTT ACT CAT-CHK₆HC (SEQ ID No. 36). In one embodiment, the invention provides a molecule comprising HAIYPRH-TTT GCT CTT ACT CAT-CHK₆HC (SEQ ID No. 37). In one embodiment, the invention provides a molecule comprising GCAT-THRPPMWSPVWP (SEQ ID No. 38).

In one embodiment of the invention, the molecule further comprising a linker moiety linking between the peptide ligand, the hydrophobic moiety, the PNA/nucleic acid sequence and the positively charge moiety. In another embodiment of the invention, the linker moiety is polyethylene glycol (PEG). In another embodiment of the invention, the molecular weight of said PEG is in the range of 2000-40,000. In another embodiment of the invention, the linker moiety is a disulfide. In another embodiment of the invention, the linker moiety is an amide. In another embodiment of the invention, the linker moiety is an amine. In another embodiment of the invention, the linker moiety is an oxyamine. In another embodiment of the invention, the linker moiety is an oxyimine. In another embodiment of the invention, the linker moiety is a morpholine. In another embodiment of the invention, the linker moiety is a thioether. In another embodiment of the invention, the linker moiety is thiourea sulfonamide. In another embodiment of the invention, the linker moiety is an ether. In another embodiment of the invention, the linker moiety is an ester. In another embodiment of the invention, the linker moiety is a carbonate. In another embodiment of the invention, the linker moiety is a carbamate. In another embodiment of the invention, the linker moiety is guanidine. In another embodiment of the invention, the linker moiety is avidin. In another embodiment of the invention, the linker moiety is strepavidin. In another embodiment of the invention, the linker moiety is biotin. In another embodiment of the invention, the linker moiety is praline. In another embodiment of the invention, the linker moiety is lysine. In another embodiment of the invention, the linker moiety is cysteine.

In one embodiment of the invention, the liable linker or peptide bond to polyethylene glycol conjugated to the molecule improves phramacokinetic properties and overcomes possible side effects induced by the amphiphilic PNA. In one embodiment of the invention, the linker is conjugated to the molecule via a known technology.

In one embodiment, the invention further provides a composition comprising as an active ingredient an effective amount of one or more molecules according to the invention, together with one or more pharmaceutically acceptable excipients or adjuvants. In one embodiment of the invention, the composition is formulated for oral or parenteral administration. In another embodiment of the invention, the composition is formulated as uncoated tablets, coated tablets, pills, capsules, powder or suspension. In another embodiment of the invention, the composition is formulated for intravenous administration. In another embodiment of the invention, the composition is formulated intranasal administration. In another embodiment of the invention, the composition is formulated administration via aerosols. In another embodiment of the invention, the composition is formulated for transdermal administration. In another embodiment of the invention, the composition is formulated in an ointment, cream or gel form. In another embodiment of the invention, the compositions of the invention are formulated in a liquid dosage form. Examples of suitable liquid dosage forms include solutions or suspensions in water, pharmaceutically acceptable fats and oils, alcohols or other organic solvents, including esters, emulsions, syrups or elixirs, solutions and/or suspensions.

Suitable excipients and carriers can be solid or liquid and the type is generally chosen based on the type of administration being used. Liposomes may also be used to deliver the composition. Examples of suitable solid carriers include lactose, sucrose, gelatin and agar. Oral dosage forms may contain suitable binders, lubricants, diluents, disintegrating agents, coloring agents, flavoring agents, flow-inducing agents, and melting agents. Liquid dosage forms may contain, for example, suitable solvents, preservatives, emulsifying agents, suspending agents, diluents, sweeteners, thickeners, and melting agents. Parenteral and intravenous forms should also include minerals and other materials to make them compatible with the type of injection or delivery system chosen.

In one embodiment, the invention provides a method for the synthesis of a molecule according to the invention.

In one embodiment, the invention further provides a method for delivering a PNA/nucleic acid sequence across a cellular membrane comprising the step of applying to a cell an effective amount of one or more molecules according to the invention. In one embodiment of the invention, the cell is an endothelial cell. In another embodiment of the invention, the cell is a neuronal cell. In another embodiment of the invention, the cell is a glial cell. In another embodiment of the invention, the cell is a muscle cell.

In one embodiment, the invention provides a method for the improved delivery of PNAs into mammalian cells. In one embodiment, the invention provides an amphiphilic PNA chimeric moiety with improved neuronal, endothelial delivery properties.

In one embodiment, the invention further provides a method for intracellular targeting of a PNA/nucleic acid sequence to an intracellular organelle comprising the step of applying to a cell an effective amount of one or more molecules according to the invention. In one embodiment of the invention, charge distribution, length of the apolar PNA/nucleic acid chain and hydrophobicity can affect sub-cellular compartization. In one embodiment, the invention provides a method for intracellular targeting of a PNA/nucleic acid sequence to an intracellular organelle comprising the step of applying to a cell an effective amount of one or more molecules of the invention, wherein the molecules cross the nuclear membrane.

In one embodiment, the invention further provides a method for delivering a PNA/nucleic acid sequence to the brain across the blood brain barrier, said method comprising the step of administering to a subject an effective amount of one or more molecules according to the invention. In another embodiment of the invention, the invention provides a method for delivering a PNA/nucleic acid sequence to the brain across the blood brain barrier, the method comprising the step of administering to a subject a composition according to the invention.

In one embodiment of the invention, polyarginine oligomers are used to improve BBB penetration of PNA-based constructs. In another embodiment of the invention, arginine guanido groups are used to improve BBB penetration of PNA-based constructs. In one embodiment of the invention, the increased brain uptake through the BBB is via guanidine basic amino acid transporters. In another embodiment of the invention, the increased brain uptake is the result of augmented AMT as a result of increased permeability surface due to the presence of positive charge.

In one embodiment of the invention, polyamines are used to improve BBB penetration of PNA-based constructs. In another embodiment of the invention, polyamine is putrescine. In another embodiment of the invention, polyamine is sperimidine. In another embodiment of the invention, polyamine is putrescine. In another embodiment of the invention, polyamine is spermine. In one embodiment of the invention, the increased brain uptake through the BBB is via polyamine transporters. In another embodiment of the invention, the increased brain uptake is the result of augmented AMT as a result of increased permeability surface due to the presence of positive charge.

In one embodiment, the invention further provides a method for delivering a gene across the blood brain barrier for expression in the brain, said method said method comprising administering to a subject an effective amount of one or more molecules according to the invention. In another embodiment of the invention, the invention provides a method for delivering a gene across the blood brain barrier for expression in the brain, said method said method comprising administering to a subject one a composition according to the invention.

In one embodiment, the invention further provides a method for modulating gene expression, said method said method comprising administering to a subject an effective amount of one or more molecules according to the invention. In another embodiment of the invention, the invention provides a method for modulating gene expression, said method said method comprising administering to a subject one a composition according to the invention.

In one embodiment, the invention provides a kit comprising an effective amount of one or more molecules according to the invention. In another embodiment of the invention, the kit allows gene labeling. In another embodiment of the invention, the kit further comprising labeling and/or reaction buffers. In another embodiment of the invention, the molecule is conjugated to a fluorescent label, a colorimetric label, a radiolabel label or a chemical label

In one embodiment, the invention further provides a method for the treatment, prevention and control of a disease, said method comprising administering to a subject an effective amount of one or more molecules according to the invention. In another embodiment of the invention, the invention provides a method for the treatment, prevention and control of a disease, said method comprising administering to a subject a composition according to the invention. In one embodiment of the invention, the disease is a central nervous system related disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control.

This invention is further illustrated in the Experimental Details section, which follows. This section is set forth to aid in an understanding of the invention but is not intended to, and should not be construed to, limit in any way the invention as set forth in the claims that follow thereafter.

### EXPERIMENTAL DETAILS SECTION

Methods

PNA and Peptide Synthesis.

Oligomers were made on a Chemspeed Automatic Synthesizer on 5micromole scale applying double coupling of fluorenylmethoxycarbonyl (Fmoc) protected nucleobases monomers or amino-acids (AA) with a ratio of 1:3 each coupling cycle. Resin used was either wang resin (0.57 mmole/g) or TGA resin 9 Novasyn, 0.15 mmole/g) already loaded with amino acid. Total reaction volume was 250 microliter and coupling reagent utilized was either BOP or HBTU in the presence of diisopropylethylamine and 2,6-lutidine. PNAs or PNAs combined with peptides were deprotected and removed from the resin by trifluoro acetic acid 1 ml containing triethylsilyl (2 h). Product was percipitate with cold diethylether 14 ml collected and subjected to RP-HPLC on a 10 micron vydac column at 50° C. Gradient 10% to 90% acetonitril (ACN) in 25 min, RT ˜10 min. Analysis: Mass-spectra: MALDI-TOF

Cell Culture:

As an in vitro models for neurons and for BBB we used the human neuroblastoma NMB cell line the rat catecholaminergic cell line PC12 and the mice brain-derived endothelial cell line bEND. NMB cells were adapted to grow in Dulbecco modified Eagle medium (DMEM) with 10% Hyclone calf serum (Simantov et al., 1996). PC12 cells were grown in DMEM, 10% horse serum and 5% calf serum. bEND3 cells were grown in DMEM, %10 calf serum supplemented with 2 mM glutamine. Cells were maintained in a 10% CO₂ humidified incubator at 37° C. Cells were routinely sub-cultured every four to five days. When cells (5×10³) were plated in 96 wells, dishes were pretreated with poly-L-ornithine.

Uptake Experiments

Uptake of PNA and conjugated PNA was determined by measurement of fluorescence retained in cells following incubation with fluorescein-labelled peptide-PNA conjugated or fluorescein-labelled unmodified PNA. The compounds were incubated with NMB, PC12 or bEND3 for different time period, After incubation, the cells were extensively washed with ice-cold PBS followed by acid wash (1.0 M NaCl/0.4 M NaOAc, PH 3.3). A procedure that was found to removed both noninternalized oligomer and dead cells. After the final wash intra-cellular fluorescence was determined by fluoremeter (FLUOstar BMG Labtechnologies).

### EXAMPLES

### Example 1

### PC12 Cells

Uptake of Conjugated PNAs into the Neuronal Cell Line PC12:

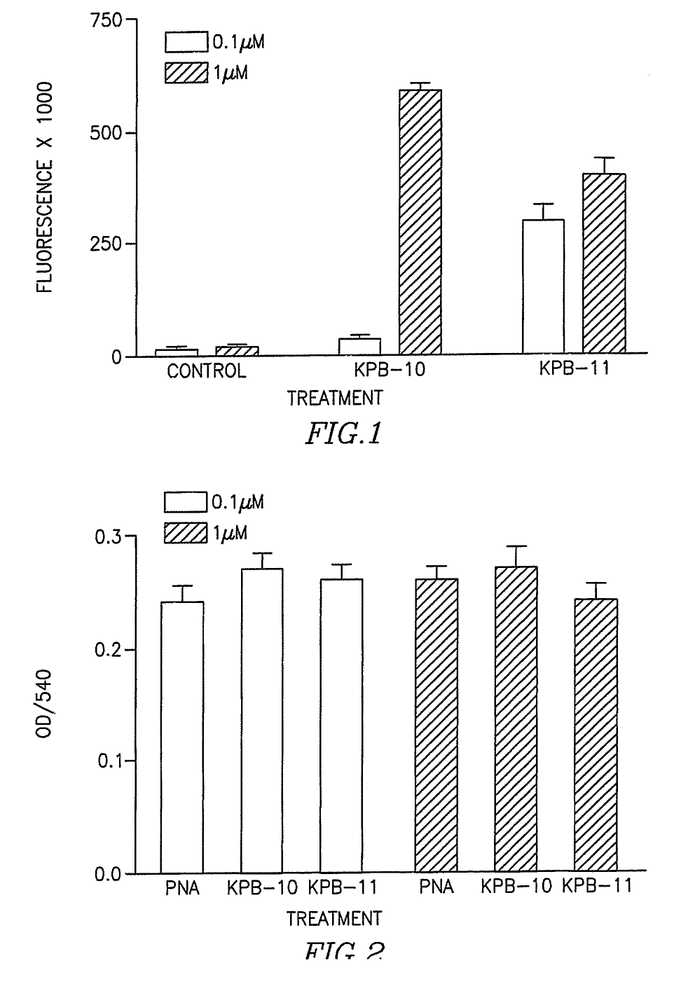

Cells were seeded on 96 well dishes coated with poly-1-lysine. Day after seeding medium was replaced with fresh medium without serum containing different concentrations (0.1-1 μM) of PNAs. Following 3 hours incubation medium was removed and cells were washed three times with acid wash solution and fluorescent determined (FIG. 1).

| | | |
|---|---|---|
| Control = | TTT GCT CTT ACT CAT | (SEQ ID No. 39) |
| KBP10 = | CHK₆HC | (SEQ ID No. 40) |
| | - TTT GCT CTT ACT CAT | (SEQ ID No. 39) |
| | - THRPPMWSPVWP | (SEQ ID No. 41) |
| KBP11 = | CHK₆HC | (SEQ ID No. 40) |
| | - TTT GCT CTT ACT CAT - | (SEQ ID No. 39) |
| | HAIYPRH | (SEQ ID No. 41) |

As can be seen from FIG. 1, the uptake of either KBP10 and KBP11 into PC12 cells was much higher than the uptake of PNA alone.
Measurement of Cellular Toxicity:

PC12 cells were incubated with PNA or peptide-PNA conjugates for 48 hours. At the end of the incubation, cell morphology was examined by light microscopy. Medium was replaced with medium containing neutral red. Neutral-red uptake was determined by spectrophotometer and was used as an index for cellular toxicity (FIG. 2).

| | | |
|---|---|---|
| Control (PNA) = | TTT GCT CTT ACT CAT | (SEQ ID No. 39) |
| KBP10 = | CHK₆HC | (SEQ ID No. 40) |
| | - TTT GCT CTT ACT CAT | (SEQ ID No. 39) |
| | - THRPPMWSPVWP | (SEQ ID No. 41) |
| KBP11 = | CHK₆HC | (SEQ ID No. 40) |
| | - TTT GCT CTT ACT CAT - | (SEQ ID No. 39) |
| | HAIYPRH | (SEQ ID No. 41) |

As can be seen from FIG. 2 there was no significant difference in the neutral red uptake to PC12 cells between the control and KBP10 and KBP11.

### Example 2

### bEND3 Cell Line BBB Cellular Model

Uptake of fluorescence labeled PNA (TTT GCT CTT ACT CAT ) (SEQ ID. No. 39) or peptide-PNA to bEND3 (CHK₆HC (SEQ ID. No. 40)-TTT GCT CTT ACT CAT-(SEQ ID. No. 39) HAIYPRH (SEQ ID. No. 41).

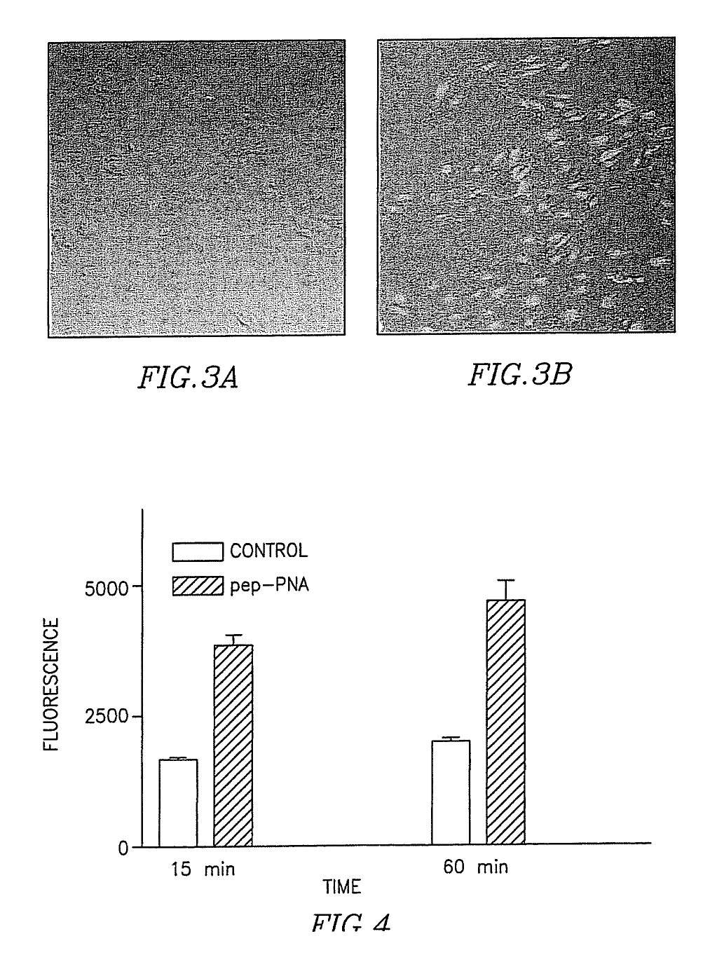

bEND3 cells were seeded on a poly-L-ornithine coated 35 mm dish. 24 hours following seeding the cell culture medium was replaced with DMEM containing 10 μM PNA or peptide-PNA. Cells were incubated for 4 hours. Following incubation cells were washed 3 times with PBS and medium was replaced with fresh DMEM cells as observed by confocal microscopy (FIG. 3). As can be clearly seen, the uptake of peptide-PNA is clearly observed whereas uptake of PNA alone is invisible.

### Example 3

### NMB Cell Line

Uptake of PNA (GCAT) or conjugated peptide PNA (GCAT-THRPPMWSPVWP) (SEQ ID. No. 42) into the human neuronal cell line NMB. Cells were seeded on 96 well dishes coated with poly-1-ornithine. One day after seeding the medium was replaced with a fresh medium without serum, containing 1 micromolar PNAs. Following 15 or 60 min. incubation medium was removed and cells were washed three times with acid wash solution. Fluorescent was determined (FIG. 4).

### Example 4

### In Vivo Brain Uptake-Intracarotid Injection

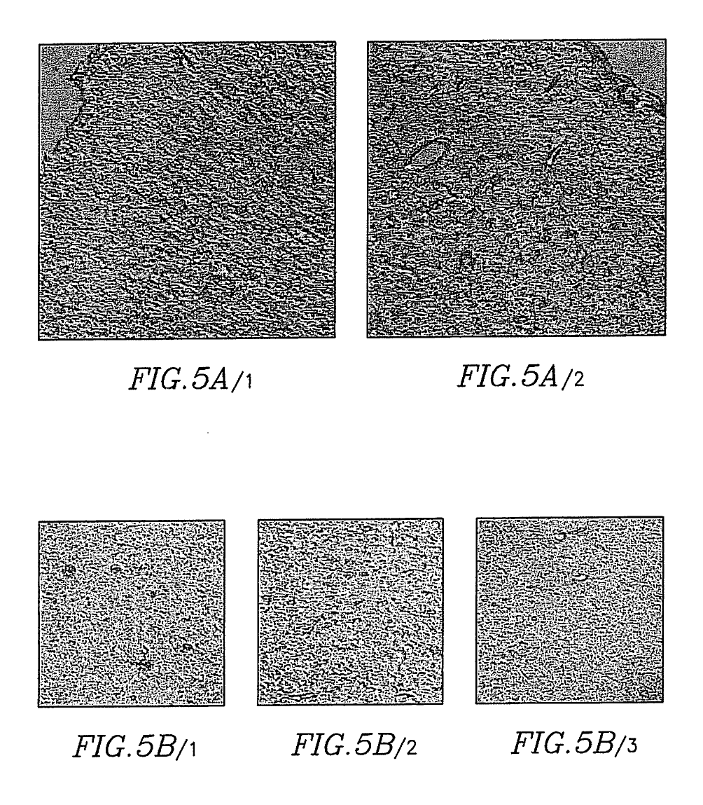

The uptake of conjugated peptide-PNAs (KBP10, 11) to the luminal side of rat brain capillaries was measured following intracarotid injection of 1 μM PNAs solution. A 300 gr Wister rat was anesthetized using Equitezine (sodium pentobarbital, chloral hydrate) 1 ml/300 gr intra-peritoneal injection. The rat was position on its back and a midline cut was preformed between the pectoral muscle and the mandible. The muscles were separated by blunt dissection and the blood vessels were exposed. The external carotid, pterigopalatine and occipital arteries were occluded using a suture and clamps were positioned on the common carotid before the bifurcation and on the internal carotid. An incision is made in the artery near the bifurcation and a suture is placed under the bifurcation to be used later. A saline pre-filled clear vinyl tube (ID 0.5 mm, OD 0.8 mm) is inserted. The suture was closed to hold the tube in the vessel and avoid lickings. A micro-infusion pump was connected to the tube and the solution was infused in a rate of 1 ml/10 min. A total of 0.5 ml was infused. The tube was retrieved, vessel occluded and the animal was perfused with PBS (×1). PBS solution was replaced and brains perfused with 4% paraformaldehyde. Brains were removed and cut to 8 μm slides for confocal microscopy analysis. Confocal microscopy analysis of FITC labelled KBP10 or KBP11 uptake following 5 minutes intracarotid injection of KBP10 (FIG. 5a) or KBP11 (FIG. 5b) show parenchymal distribution of KEP10 or KEP11 indicating that compound significantly cross the BBB.

1. A molecule represented by any one of the formulas I-IV:
[(L)ₜ-(N)ᵣ-(H)_{q}-(P)ₛ]ₓ I.
[(L)ₜ-(H)_{q}-(N)ᵣ-(P)ₛ]ₓ II.
[(P)ₛ-(N)ᵣ-(H)_{q}-(L)ₜ]ₓ III.
[(P)ₛ-(H)_{q}-(N)ᵣ-(L)ₜ]ₓ IV.
wherein N is a nucleic acid sequence in a length of 1-100 bases, L is a peptide ligand which binds to a specific receptor and P is a positively charge moiety and H is a hydrophobic moiety; and
wherein r is an integer of 1-25, t is an integer of 1-50, s is an integer of 1-25, q is an integer of 0-20, and x is an integer of 1-20.

2. The molecule according to claim 1, wherein said nucleic acid sequence is a mRNA, a cDNA, a DNA, a DNA analog, a polyamide nucleic acid (PNA), a PNA morpholino, an aminoethylprolyl (aep) PNA, a pyrrolidinyl PNA, an oligonucleotide, an oligonucleotide analog, a ribozyme or an RNAi.

3. The molecule according to claim 1, wherein said nucleic acid sequence is a PNA.

4. The molecule according to claim 1, wherein said nucleic acid sequence is an antisense, an antigene or a decoy function.

5. The molecule according to claim 1, wherein said nucleic acid sequence is neutral or negatively charged.

6. The molecule according to claim 1, wherein said peptide ligand binds a receptor to transferrin, insulin, insulin growth factor, or leptin.

7. The molecule according to claim 6, wherein said insulin growth factor is insulin growth factor-I or insulin growth factor-II.

8. The molecule according to claim 1, wherein said peptide ligand is HAIYPRH (SEQ ID. No. 1) or THRPPMWSPVWP (SEQ ID. No. 2).

9. The molecule according to claim 1, wherein said hydrophobic moiety is a nucleic acid.

10. The molecule according to claim 1, wherein said hydrophobic moiety is hydrophobic peptides, lipid acid, lipid molecules, octanol, cholesterol, hydrophobic peptide protecting group, adamantine, pyrene, eicosenoic acid, C₍₆₋₁₆₎ glyceride lipid, phenoxazine, DMT group, cholenic acid, lithocholic acid, myristic acid, palmitic acid, heptadecyl group, hexadecylglycerol, geranyloxyhexyl group, hexadecylamine, dihydrotestosterone, 1-pyrene butyric acid, alkanoic acid, alkanol or any derivatives thereof.

11. The molecule according to claim 10, wherein said alkanoic acid is represented by the structure R—(CH₂)n-COOH, wherein n=1-20 and R is a linear or branched alkyl.

12. The molecule according to claim 10, wherein said alkanol is represented by the structure R—(CH₂)n-OH, wherein n=1-20 and R is a linear or branched alkyl.

13. The molecule according to claim 10, wherein said lipid acid is undecanoic acid and/or docosahexanenonic acid.

14. The molecule according to claim 10, wherein said hydrophobic peptide protecting group is Fmoc or Tboc.

15. The molecule according to claim 1, wherein said positively charge moiety is a nucleic acid.

16. The molecule according to claim 1, wherein said positively charge moiety is positively charge peptide, peptidomimetic, polycations, histidine, imidazole group, 2-O-aminopropyl, 2-O-dimethylaminopropyl, 2-O-imidazolyl-ethyl, 2-O-aminoethylamino-oxyethyl, 2-dimethylaminoethyl-oxyethyl or any derivative thereof.

17. The molecule according to claim 1, wherein said positively charge moiety comprises at least one group of arginine, polyamin and/or guanidine.

18. The molecule according to claim 17, wherein said polyamine is spermine, spermidine or putricine.

19. The molecule according to claim 1, wherein said peptide ligand, said nucleic acid sequence, said hydrophobic moiety, and said positively charge moiety are linked to each other directly via peptide bonds.

20. The molecule according to claim 1, further comprising a linker moiety linking between said peptide ligand, said hydrophobic moiety, said nucleic acid sequence and said positively charge moiety.

21. The molecule according to claim 20, wherein said linker moiety is polyethylene glycol, disulfide, amide, amine, oxyamine, oxyimine, morpholine, thioether, thiourea sulfonamide, ether, ester, carbonate, carbamate, avidin, strepavidin, biotin, praline, lysine, cysteine, guanidine or any combination thereof.

22. The molecule according to claim 21, wherein the molecular weight of said polyethylene glycol is in the range of 2000-40,000.

23. A molecule represented by any one of the formulas V-VIII:
[(L)ₜ-(PNA)ᵣ-(H)_{q}-(P)ₛ]ₓ V.
[(L)ₜ-(H)_{q}-(PNA)ᵣ-(P)ₛ]ₓ VI.
[(P)ₛ-(PNA)ᵣ-(H)_{q}-(L)ₜ]ₓ VII.
[(P)ₛ-(H)_{q}-(PNA)ᵣ-(L)ₜ]ₓ VIII.
wherein the length of said PNA sequence is 1-100 bases, L is a peptide ligand which binds to a specific receptor and P is a positively charge moiety, and H is a hydrophobic moiety; and
wherein r is an integer of 1-25, t is an integer of 1-50, s is an integer of 0-25, q is an integer of 0-20 and x is an integer of 1-20.

24. The molecule according to claim 23, wherein said PNA sequence is an antisense, an antigene or a decoy function.

25. The molecule according to claim 23, wherein said PNA sequence is neutral or negatively charged.

26. The molecule according to claim 23, wherein said peptide ligand binds a receptor to transferrin, insulin, insulin growth factor, Insulin growth factor or leptin.

27. The molecule according to claim 26, wherein said insulin growth factor is insulin growth factor-I or insulin growth factor-II.

28. The molecule according to claim 23, wherein said peptide ligand is HAIYPRH (SEQ ID. No. 1) or THRPPMWSPVWP (SEQ ID. No. 2).

29. The molecule according to claim 23, wherein said hydrophobic moiety is a nucleic acid.

30. The molecule according to claim 23, wherein said hydrophobic moiety is hydrophobic peptides, lipid acid, lipid molecules, octanol, cholesterol, hydrophobic peptide protecting group, adamantine, pyrene, eicosenoic acid, C₍₆₋₁₆₎ glyceride lipid, phenoxazine, DMT group, cholenic acid, lithocholic acid, myristic acid, palmitic acid, heptadecyl group, hexadecylglycerol, geranyloxyhexyl group, hexadecylamine, dihydrotestosterone, 1-pyrene butyric acid, alkanoic acid, alkanol or any derivatives thereof.

31. The molecule according to claim 30, wherein said alkanoic acid is represented by the structure R—(CH₂)n-COOH, wherein n=1-20 and R is a linear or branched alkyl.

32. The molecule according to claim 30, wherein said alkanol is represented by the structure R—(CH₂)n-OH, wherein n=1-20 and R is a linear or branched alkyl.

33. The molecule according to claim 30, wherein said lipid acid is undecanoic acid and/or docosahexanenonic acid.

34. The molecule according to claim 30, wherein said hydrophobic peptide protecting group is Fmoc or Tboc.

35. The molecule according to claim 23, wherein said positively charge moiety is a nucleic acid.

36. The molecule according to claim 23, wherein said positively charge moiety is positively charge peptide, peptidomimetic, polycations, histidine, imidazole group, 2-O-aminopropyl, 2-O-dimethylaminopropyl, 2-O-imidazolyl-ethyl, 2-O-aminoethylamino-oxyethyl, 2-dimethylaminoethyl-oxyethyl or any derivative thereof.

37. The molecule according to claim 23, wherein said positively charge moiety comprises at least one group of arginine, polyamin and/or guanidine.

38. The molecule according to claim 37, wherein said polyamine is spermine, spermidine or putricine.

39. The molecule according to claim 23, wherein said peptide ligand, said PNA sequence, said hydrophobic moiety and said positively charge moiety are linked to each other directly via peptide bonds.

40. The molecule according to claim 23, further comprising a linker moiety linking between said peptide ligand, said hydrophobic moiety, said PNA sequence and said positively charge moiety.

41. The molecule according to claim 40, wherein said linker moiety is polyethylene glycol, disulfide, amide, amine, oxyamine, oxyimine, morpholine, thioether, thiourea sulfonamide, ether, ester, carbonate, carbamate, avidin, strepavidin, biotin, praline, lysine, cysteine, guanidine or any combination thereof.

42. The molecule according to claim 41, wherein the molecular weight of said polyethylene glycol is in the range of 2000-40,000.

43. A composition comprising as an active ingredient an effective amount of one or more molecules according to claim 1, together with one or more pharmaceutically acceptable excipients or adjuvants.

44. A composition comprising as an active ingredient an effective amount of one or more molecules according to claim 23, together with one or more pharmaceutically acceptable excipients or adjuvants.

45. The composition according to claim 43, formulated for oral or parenteral administration.

46. The composition according to claim 44, formulated for oral or parenteral administration.

47. The composition according to claim 43, formulated as uncoated tablets, coated tablets, pills, capsules, powder or suspension.

48. The composition according to claim 44, formulated as uncoated tablets, coated tablets, pills, capsules, powder or suspension.

49. The composition according to claim 43, formulated for intravenous administration.

50. The composition according to claim 44, formulated for intravenous administration.

51. A method for delivering a nucleic acid sequence across a cellular membrane comprising the step of applying to a cell an effective amount of one or more molecules according to claim 1.

52. The method according to claim 51, wherein said cell is an endothelial cell, neuronal cell or glial cell.

53. A method for delivering a PNA sequence across a cellular membrane comprising the step of applying to a cell an effective amount of one or more molecules according to claim 23.

54. The method according to claim 53, wherein said cell is an endothelial cell, neuronal cell or glial cell.

55. A method for delivering a nucleic acid sequence to the brain across the blood brain barrier, said method comprising the step of administering to a subject an effective amount of one or more molecules according to claim.

56. A method for delivering a PNA sequence to the brain across the blood brain barrier, said method comprising the step of administering to a subject an effective amount of one or more molecules according to claim 23.

57. A method for delivering a nucleic acid sequence to the brain across the blood brain barrier, said method comprising the step of administering to a subject a composition according to claim 43.

58. A method for delivering a PNA sequence to the brain across the blood brain barrier, said method comprising the step of administering to a subject a composition according to claim 44.


SEQUENCE LISTING (18 sequences)

SEQ ID NO: 1 - protein, 7 aa, Homo sapiens
HAIYPRH

SEQ ID NO: 2 - protein, 12 aa, Homo sapiens
THRPPMWSPVWP

SEQ ID NO: 3 - protein, 10 aa, Homo sapiens
CHKKKKKKHC

SEQ ID NO: 4 - protein, 10 aa, Homo sapiens
CKKKKHKKKC

SEQ ID NO: 5 - protein, 10 aa, Homo sapiens
CHKKKHKKHC

SEQ ID NO: 6 - protein, 10 aa, Homo sapiens
CHKHKHKHKC

SEQ ID NO: 7 - protein, 10 aa, Homo sapiens
CHKHKHHKHC

SEQ ID NO: 8 - DNA, 8 bp, Homo sapiens
ccgctccg

SEQ ID NO: 9 - DNA, 12 bp, Homo sapiens
catggtggacgt

SEQ ID NO: 10 - DNA, 14 bp, Homo sapiens
ctttctccttttcc

SEQ ID NO: 11 - DNA, 16 bp, Homo sapiens
tactcatgggcacact

SEQ ID NO: 12 - DNA, 15 bp, Homo sapiens
tttgctcttactcat

SEQ ID NO: 13 - DNA, 4 bp, Homo sapiens
gcat

SEQ ID NO: 14 - protein, 37 aa, Unknown
  synthetic
CHKKKKKKHCTTTGCTCTTACTCATTHRPPMWSPVWP

SEQ ID NO: 15 - protein, 32 aa, Unknown
  synthetic
CHKKKKKKHCTTTGCTCTTACTCATHAIYPRH

SEQ ID NO: 16 - protein, 37 aa, Unknown
  synthetic
THRPPMWSPVWPTTTGCTCTTACTCATCHKKKKKKHC

SEQ ID NO: 17 - protein, 32 aa, unknown
  synthetic
HAIYPRHTTTGCTCTTACTCATCHKKKKKKHC

SEQ ID NO: 18 - protein, 16 aa, Unknown
  synthestic
GCATTHRPPMWSPVWP